(19) United States
(12) Patent Application Publication
(10) Pub. No.: US 2014/0079191 A1
(43) Pub. Date: Mar. 20, 2014

(54) IMAGING DEVICE WITH A MOVABLE GANTRY

(71) Applicants: Arnulf Daum, Bayreuth (DE); Ulrich Foerner, Ebensfeld (DE); Bastian Rackow, Erlangen (DE)

(72) Inventors: Arnulf Daum, Bayreuth (DE); Ulrich Foerner, Ebensfeld (DE); Bastian Rackow, Erlangen (DE)

(21) Appl. No.: 14/032,371

(22) Filed: Sep. 20, 2013

(30) Foreign Application Priority Data
Sep. 20, 2012 (DE) 102012216858.3

(51) Int. Cl. A61B 6/00

(52) U.S. Cl. CPC A61B 6/4405
USPC 378/198

(57) ABSTRACT

An imaging device has a gantry that is movable along a floor, and a supply unit for transmission of power and/or data between a stationary supply source and the movable gantry. The transmission of the power and/or data takes place via wireless transmission elements. This wireless transmission of the power and/or data between the transmission elements can take place via conductor structures, and the wireless transmission elements are arranged in the floor of the room of the imaging device and in a sled of the movable gantry.

### BACKGROUND OF THE INVENTION

1. Field of the Invention

The invention concerns an imaging device with a movable gantry.

2. Description of the Prior Art

As used herein, a movable gantry (sliding gantry) means a module that has at least one housing with a tunnel proceeding therethrough (for instance an annular (gantry) housing) and modules incorporated into the housing that enable image data acquisition. The entire gantry housing is movable on rails recessed into the floor. For image acquisition purposes, the gantry is moved over a patient bed associated with the gantry such that the patient bed projects into the tunnel.

Such movable gantries are presently used primarily in (x-ray) computed tomography systems (CT systems). An essentially annular mount (also designated as a ring mount) is supported inside the housing. On this mount, an x-ray source and an x-ray detector are attached radially opposite each other. This mount rotates around the tunnel in a known manner to acquire slice images (tomograms) of a patient on the patient bed.

In a typical image acquisition procedure, projection images of a subject to be imaged (in particular of the patient borne on the patient bed) are acquired in slices during successive feed of the gantry along the tunnel axis and are electronically stored as image data. Ultimately, slice images or three-dimensional volume data sets of the subject to be imaged are calculated by numerical back-projection of the acquired projection images at a control and evaluation computer associated with the gantry. The acquisition of a series of projection images is also designated as a scan in the following.

Such a movable gantry is frequently used in connection with an operation on the patient wherein an image acquisition should take place in the course of the operation and a dedicated CT table cannot be used.

To operate the CT system it is necessary to supply the movable gantry with power and to transfer data (in particular image data acquired in a scan) as well as control signals between the gantry and the control and evaluation computer.

In a conventional design, the lines for the power and data are directed from the gantry into what is known as a ceiling module via a cable column. The ceiling module is a housing, usually oblong that is arranged in proximity to the ceiling of an operating room, for example. This housing is arranged approximately parallel to the movement path of the gantry. A device that is designated in the following as a power routing chain is accommodated inside the housing.

Such a known power routing chain is a flexible component that directs and protects a conduit accommodated therein, with the power routing chain establishing a connection between the stationary supply source and a connection point at the positionally variable gantry.

### SUMMARY OF THE INVENTION

An object of the present invention is to optimize the imaging examination volume with a movable gantry with regard to the transmission of power and/or data between the stationary supply source and the movable gantry.

The imaging device, such as a medical imaging device, according to the invention has a gantry movable along the a floor, as well as a supply unit for the transmission of power and/or data between a stationary supply source and the movable gantry.

The invention is based on the realization that the flexibility of the gantry can be increased and the mechanical load on the cabling can be avoided by the transmission of power and/or data between the stationary supply source and the movable gantry by wireless transmission techniques.

By the use of wireless transmission elements (such as a transmission device and a reception device), a wireless solution is realized for the transmission of power and/or data. The space requirements are thereby optimized because lines for power and data must no longer by routed from the gantry into the ceiling module via a cable column; on the other hand, no mechanical loads on the cabling (which can have the consequence of a cable break) arise either.

In a preferred embodiment of the imaging device, the transmission of power and/or data between the transmission elements takes place by means of slip contacts. For example, in this way data can be transferred from the movable gantry to the stationary part of the imaging device in order to be processed further there. Such a system has at least on transmission element with a radio-frequency line connected with a transmitter as a transmission antenna, which transmission antenna is arranged at a specific location of the movable gantry so that the transmission element can interact with a second transmission element that is arranged at a specific counter-position at the stationary part of the imaging device. This second transmission element includes a receiver and at least one reception antenna connected with the receiver, which is formed by a segment of a radio-frequency line. If, in the operation of the imaging device, the transmission antenna moves at slight clearance past the reception antenna attached to the stationary part, the signals propagating on the transmitting radio-frequency line can thus be capacitively injected into the reception antenna via the developing wave.

In a further preferred embodiment of the imaging device, the transmission of the power and/or the data between the transmission elements takes place via conductor structures. For data transmission, such a system has at least two differential conductor traces (runs) arranged as mirror images of one another, these differential conductor traces being alternately charged with different polarized voltage, with the application of the one polarity corresponding to a binary zero and the application of the opposite polarity corresponding to a binary one.

In a further embodiment of the imaging device, the transmission of the data between the transmission elements takes place via radio transmission such as WLAN, Bluetooth, WiMAX or similar wireless technologies.

In an embodiment, the wireless transmission elements are arranged in the floor of the room of the imaging device, as well as in the sled of the movable gantry. In this way, power and/or data can be transmitted wirelessly between a floor element and a sled element.

In a further embodiment, the wireless transmission elements are arranged on the floor of the room of the imaging device, as well as in a sled of the movable gantry. In this way, power and/or data can be transmitted wirelessly between a floor element and a sled element.

In a further embodiment the wireless transmission elements are arranged in a wall of the room of the imaging device, as well as in a sled of the movable gantry. In this way, power and/or data can be transmitted wirelessly between a wall element and a sled element.

### BRIEF DESCRIPTION OF THE DRAWINGS

FIG. 1 shows an imaging device according to the invention with wireless data transmission.

FIG. 2 shows a further embodiment of an imaging device according to the invention with wireless data transmission.

FIG. 3 shows a third embodiment of an imaging device according to the invention with wireless data transmission.

### DESCRIPTION OF THE PREFERRED EMBODIMENTS

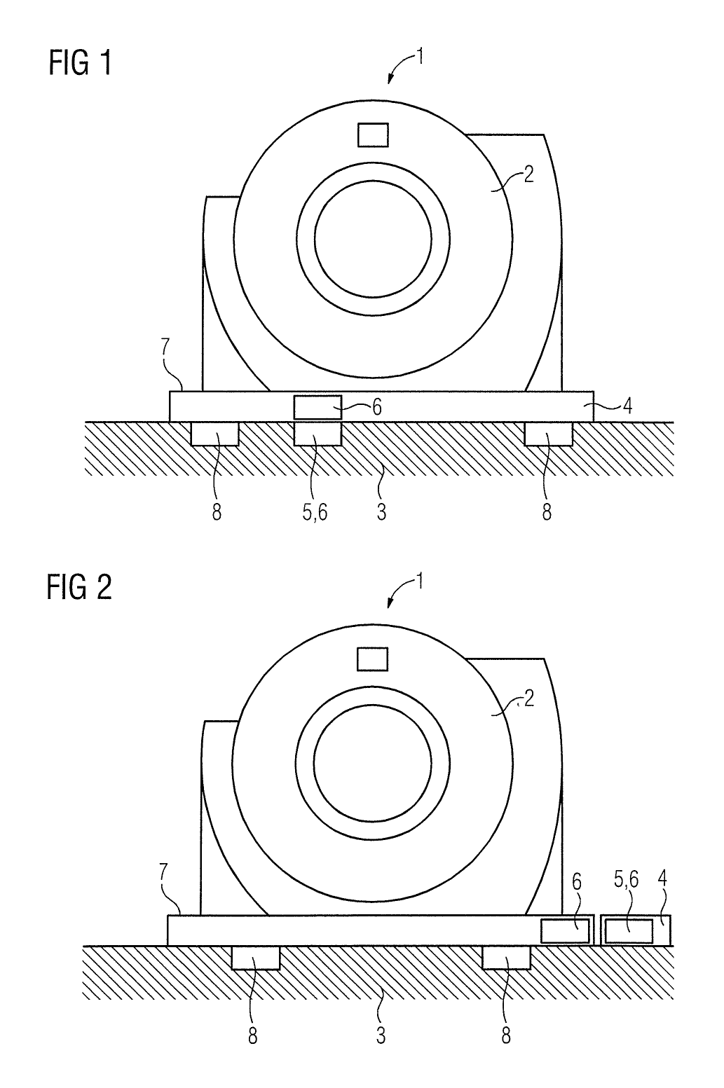

FIG. 1 shows an imaging device 1 according to the invention in which a wireless data transmission is realized via transmission elements 6 in the floor 3 of the room of the imaging device 1, as well as in a sled 7 of the movable gantry 2. Power and/or data can in this way be transmitted wirelessly between a floor element and a sled element.

In this example, the type of data transmission takes place via slip contacts. Such an arrangement has at least one transmission element 6 with a radio-frequency line connected as a transmission antenna with a transmitter, which transmission antenna is arranged at a defined location of the movable gantry 2 so that the transmission element 6 can interact with a second transmission element 6 that is arranged at a defined counter-position at the stationary part of the imaging device 1. This second transmission element 6 includes a receiver and at least one reception antenna connected with the receiver, which reception antenna is formed by a segment of a radio-frequency line. If, in operation of the imaging device 1, the transmission antenna moves at a slight distance past the reception antenna attached to the stationary part, the signals propagating on the transmitting radio-frequency line can be capacitively injected into the reception antenna via the developing wave.

FIG. 2 shows an imaging device 1 according to the invention in which a wireless data transmission is realized via transmission elements 6 on the floor 3 of the room of said imaging device 1, and in a sled 7 of the movable gantry 2. In this way, power and/or data can be transmitted wirelessly between a floor element and a sled element.

In this example, the type of data transmission takes place via conductor structures. Such an arrangement has transmission elements 6 with at least two differential conductor traces arranged in mirror-image with one another, which conductor traces are alternately charged with different polarized voltage, wherein the application of the one polarity corresponds to a binary zero and the application of the opposite polarity corresponds to a binary one.

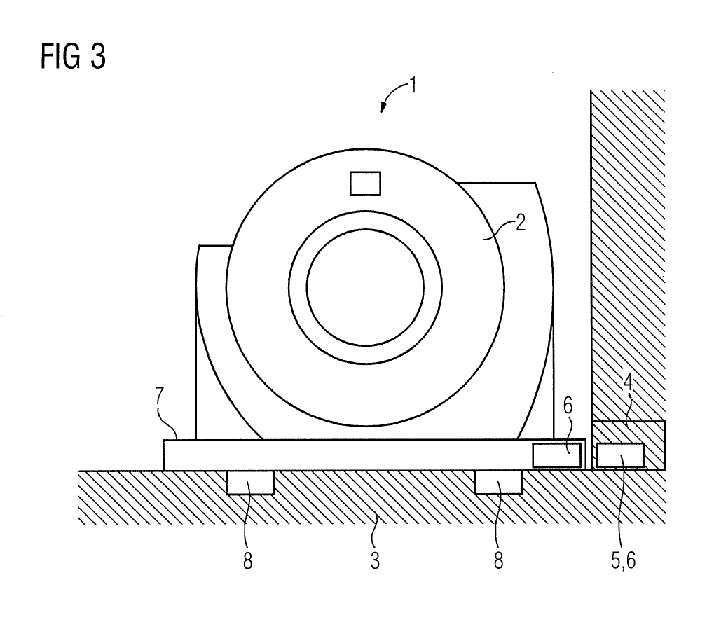

FIG. 3 shows an imaging device 1 according to the invention in which a wireless data transmission is realized via transmission elements 6 in a wall of the room of the imaging device 1, and in a sled 7 of the movable gantry 2. In this way, power and/or data can be transmitted wirelessly between a wall element and a sled element.

In this example, the type of data transmission takes place via WLAN, Bluetooth, WiMAX or similar wireless technologies.

It is common to all wireless transmission technologies shown in the figures that the space requirements are optimized on the one hand, in that conductors for power and data must no longer be routed from the gantry 2 via a cable column (for example into a ceiling module), and on the other hand in that no mechanical loads on the cables arise either, which could have the consequence of a cable break, for instance.

In summary, the invention concerns an imaging device with a gantry movable along a floor and a supply unit to transmit power and/or data between a stationary supply source and the movable gantry. The transmission of the power and/or the data takes place via wireless transmission elements. In a preferred use case, this wireless transmission of the power and/or data between the transmission elements takes place via conductor structures, and the wireless transmission elements are arranged in the floor of the room of the imaging device, as well as in a sled of the movable gantry.

Although modifications and changes may be suggested by those skilled in the art, it is the intention of the inventors to embody within the patent warranted hereon all changes and modifications as reasonably and properly come within the scope of their contribution to the art.

We claim as our invention:

1. An imaging device comprising:
a gantry configured to be movable along a floor of a room in which the gantry is installed;
a supply system that transmits at least one supplied item, selected from the group consisting of power and data from a stationary supply source for said supplied item, and said movable gantry; and
said supply system comprising wireless transmission elements configured to wirelessly transmit said at least one supplied item from said supply source to said movable gantry.

2. An imaging device as claimed in claim 1 wherein said gantry comprises a rotatable gantry component and stationary gantry component, and wherein said wireless transmission elements comprise slipperings configured to communicate said at least one supplied item at least from said stationary component to said rotatable component.

3. An imaging device as claimed in claim 1 wherein said wireless transmission elements comprise conductor runs.

4. An imaging device as claimed in claim 1 wherein said wireless transmission elements comprise radio transmission elements configured to transmit said at least one supplied item by radio transmission.

5. An imaging device as claimed in claim 1 wherein said movable gantry comprises a sled on which said movable gantry slides on said floor, and wherein said wireless transmission elements are located in said floor and in said sled.

6. An imaging device as claimed in claim 1 wherein said movable gantry comprises a sled on which said movable gantry slides on said floor, and wherein said wireless transmission elements are located on said floor and in said sled.

7. An imaging device as claimed in claim 1 wherein said room in which said movable gantry is installed comprises a wall, and wherein said movable gantry comprises a sled on which said movable gantry slides on said floor, and wherein said wireless transmission elements are arranged in said wall and in said sled.
